(19) United States
(12) Patent Application Publication
(10) Pub. No.: US 2013/0172682 A1
(43) Pub. Date: Jul. 4, 2013

(54) Tissue Retractor Assembly

(71) Applicants: Jeffrey Ransden, Fairfield, CT (US); Gregor Weaver, Waterbury, CT (US); Adam Lehman, Northford, CT (US); Vincent Mata, Monroe, CT (US)

(72) Inventors: Jeffrey Ransden, Fairfield, CT (US); Gregor Weaver, Waterbury, CT (US); Adam Lehman, Northford, CT (US); Vincent Mata, Monroe, CT (US)

(73) Assignee: YALE UNIVERSITY, New Haven, CT (US)

(21) Appl. No.: 13/704,640

(22) PCT Filed: Jun. 29, 2011
(86) PCT No.: PCT/US11/42353

§ 371 Date: Mar. 21, 2013

Related U.S. Application Data
(60) Provisional application No. 61/398,657, filed on Jun. 29, 2010.
(60) Provisional application No. 61/398,612, filed on Jun. 29, 2010.
(60) Provisional application No. 61/398,633, filed on Jun. 29, 2010.
(60) Provisional application No. 61/398,645, filed on Jun. 29, 2010.

(51) Int. Cl. A61B 1/32

(52) U.S. Cl. CPC A61B 1/32
USPC 600/204

(57) ABSTRACT

A tissue retractor assembly is provided having a cannula which houses a grasper and an anchor, the grasper extended axially from the cannula, the grasper having legs for gripping tissue and a locking ring for locking the legs, and an anchor having sharpened legs, the grasper being secured and adjusted relative to the anchor by a length of suture. Another tissue retractor assembly is provided having a cannula which houses an anchor, a wire form and a grasper, the wire form a coiled spring which expands radially, and a grasper for pulling tissue through the form. A further retractor assembly is provided having a cannula which houses an anchor and a grasper, the grasper a loop of suture configured to grasp and tighten around tissue. Another tissue retractor assembly is provided having a cannula which houses a first and second graspers, the graspers defined by C-shaped clips.

### CROSS-REFERENCE TO RELATED APPLICATIONS

This application claims the benefit of co-pending, commonly assigned U.S. Provisional Patent Application Nos. 61/398,612, 61/398,633, 61/398,645 and 61/398,657, each of which was filed on Jun. 29, 2010. The entire contents of the foregoing provisional patent applications are incorporated herein by reference.

### BACKGROUND

1. Technical Field

The present disclosure is directed generally to tissue retractor assemblies and, more particularly, to tissue retractor assemblies for single incision laparoscopic surgery.

2. Background Art

Single port laparoscopic surgery is a surgical procedure that may provide fewer risks, less patient trauma and/or reduced surgical time. In a typical single port procedure, a port is introduced through the umbilicus to gain access to internal organs and/or desired anatomical region(s). Retraction of the gall bladder or other organs is generally required during single port procedures. However, retraction is difficult with single port access because the port location is often caudal to the organs and provides limited access for an additional retraction instrument. Retraction is also an important issue in other port-based procedures, even when multiple ports are employed. Thus, tissue and/or organ retraction is generally required in a host of surgical procedures including, enter alia, gall bladder, appendix, colon, bariatric, hysterectomy and other surgical procedures.

Thus, a need exists for an organ retraction system that may be delivered in a minimally invasive manner, e.g., through a 5 mm laparoscopic port, without requiring additional abdominal incision(s) and/or ports to facilitate introduction of the organ retraction system. A further need exists for an organ retraction system that is atraumatic, e.g., reducing the risk of organ damage and/or puncture in connection with tissue engagement and/or retraction. Reduction in such risks is important because, inter alia, organ trauma and/or puncture may cause infection, e.g., release of bile from the gall bladder may cause infection in the peritoneal space and increase patient risk. An additional need exists for an organ retraction system wherein the tension or traction of the organ can be adjusted during the procedure extracorporeally, e.g., without removing and/or reintroducing the delivery device. Still further, a need exists for an organ retraction system which can grasp varied anatomical presentations of target organs. These and other needs are addressed by the assemblies of the present disclosure.

### SUMMARY

In accordance with embodiments of the present disclosure, tissue retractor assemblies are disclosed that are particularly advantageous for use in single port and multi-port laparoscopic surgery procedures or similar operations. Generally, the disclosed tissue retractor assemblies are laparoscopic surgical assist devices which facilitate the retraction of various organs and/or structures intracorporeally. In exemplary embodiments, the tissue retractor is a multi-component device configured and dimensioned to be delivered through a 5 mm laparoscopic or other minimally invasive access device, and provides an atraumatic means to grasp and hold an organ or other anatomical structure, e.g., the gall bladder.

In accordance with embodiments of the present disclosure, exemplary tissue retractor assemblies are disclosed which are adapted to retract an organ or other anatomical structure through cooperative interaction between an atraumatic grasper and an anchored guide member/suture subassembly, e.g., a suture that passes through an anchor positioned or otherwise secured with respect to a fixed position (e.g., the abdominal wall). More particularly, the disclosed tissue retractor assemblies may function by (i) placing or securing an anchor with respect to an anatomical structure, e.g., the abdominal wall, (ii) associating a suture with the anchor (either before or after securing the anchor with respect to the anatomical structure), (iii) engaging, attaching and/or securing a deployable atraumatic grasper with respect to an organ, tissue or other anatomical structure, and (iv) managing/manipulating the suture that is attached or otherwise secured with respect to the grasper and routed through the anchor, thereby allowing the organ/tissue/anatomical structure to be retracted, moved or otherwise manipulated, e.g., by tensioning the suture. Of note, the suture is advantageously passed through the abdominal wall, e.g., through a port, and is generally manipulated by a surgeon/user from such external location.

In exemplary implementations, the tissue retractor assembly is adapted for introduction and use through an abdominal wall, e.g., through a 5 mm port, and includes a cannula that defines an axis and a distal end. A grasper and an anchor are detachably secured with respect to the distal end of the cannula. A suture is cooperatively associated with the grasper and the anchor for movement/manipulation of the grasper relative to the anchor. The grasper generally is configured and dimensioned to be extended axially from the cannula, and includes first and second legs for gripping an organ, tissue and/or other structure. The grasper further includes a tubular member that is axially movable so as to move the legs/jaws of the grasper relative to each other, e.g., through a cinching or ramming action. The tubular member may function as or otherwise cooperate with a locking mechanism that is configured and dimensioned to be advanced/pushed distally to grasp and lock the first and second leg/jaw in engagement with a target structure, e.g., organ, tissue and/or other structure.

The anchor generally is configured and dimensioned to be deployed from the distal end of the cannula. The anchor may be advantageously secured relative to an anatomical location/structure within the abdominal cavity, e.g., the anchor may be attached to an abdominal wall adjacent to an organ, tissue or other structure of interest. The anchor is generally defined by a body, e.g., a cylindrical body, and at least two sharpened legs that extend relative thereto. In exemplary embodiments, the sharpened legs are fabricated from a resilient material, e.g., nitinol or stainless steel, and formed/shaped so as to resiliently move between first and second positions/orientations. More particularly, the legs are advantageously fabricated so as to move between first positions (e.g., during abdominal introduction) and second positions (e.g., after deployment through the cannula within the abdominal cavity) to provide anchoring functionality. Thus, the at least two legs may automatically deploy into the abdominal wall, e.g., in a substantially crossing configuration, to effect fixation of the anchor relative to the abdominal wall. The sharpened ends of the legs facilitate tissue penetration and the arcuate orientation upon deployment resists withdrawal of the anchor from the abdominal wall.

According to the present disclosure, a suture may be introduced to the abdominal cavity, e.g., through the cannula, and passed through (i) the body of the anchor (or through an extension associated with the body) and (ii) a cooperative aspect of the grasper. Typically, the suture is pre-associated with the grasper and anchor before abdominal introduction. The suture may be manipulated by the surgeon/user from a position external to the abdominal cavity and, based upon passage through the anchor, a desired level of tension and/or directional force may be delivered to the grasper. Thus, in exemplary implementations, the position of the grasper relative to the anchor may be remotely adjusted through suture manipulation, e.g., by introducing an additional length of suture to the abdominal cavity or withdrawing a length of suture from the abdominal cavity.

The disclosed tissue retractor assembly, therefore, includes an anchor which provides a robust attachment to a desired substrate, e.g., a peritoneal structure associated with the abdominal wall, that is not possible with non-piercing anchors. In addition, the disclosed tissue retractor assembly may be used to transfer a force to a grasper positioned within the abdominal cavity, e.g., through manipulation of a length of suture or fiber that extends from the abdominal cavity, thereby enabling a surgeon/user to tension and/or reposition the grasper relative to a substantially fixed point defined by the anchor. In exemplary implementations, the suture/fiber passes through a port to effectuate and/or support minimally invasive surgical procedures. The disclosed assembly thus enables introduction and manipulation of an advantageous grasper through a minimally invasive point of access, e.g., a 5 mm port.

In accordance with further embodiments of the present disclosure, the first and second legs/jaws of the grasper are fabricated from stainless steel or other material providing the requisite strength/resilience. The legs/jaws are generally formed into a desired initial shape. At least one of the first and second legs/jaws of the grasper may further include serrations on a distal end thereof in order to better grasp and/or capture tissue. The locking mechanism associated with the grasper may be defined by a locking ring that is configured and dimensioned to be pushed or otherwise advanced distally relative to the grasper so as to pass over/around proximal portions of the first and second legs/jaws, thereby causing the first and second legs/jaws to clinch shut over the tissue, organ or other anatomical structure, e.g., through a camming action effectuated by the distally locking ring.

In accordance with further embodiments of the present disclosure, one or both of the first and second legs/jaws of the grasper may include a rubber or other coating applied to the distal ends thereof (in whole or in part) in order to better grasp and capture tissue in an atraumatic manner.

In accordance with further embodiments of the present disclosure, an exemplary tissue retractor assembly is disclosed that is adapted to (i) fire an anchor with a retractable sharp tip and retractable barbs into an abdominal wall or other anatomical structure, (ii) deploy a wire form to grasp an organ or other structure to be retracted or otherwise manipulated, and (iii) facilitate management of a suture that is attached or otherwise secured relative to the wire form and is routed through the anchor, thus allowing the organ/structure to be retracted or manipulated by tensioning the suture. Specifically, an exemplary tissue retractor assembly includes a cannula which is configured and dimensioned to house or otherwise detachably secure an anchor, a wire form and optionally a grasper. The anchor is configured and dimensioned to be deployed from the cannula and attached relative to an abdominal wall adjacent to an organ or other structure of interest, e.g., anterior thereto.

The anchor may be defined by an outer tube and a central shaft, and may further include at least two barbs which are configured to be deployed when the central shaft is pulled axially. The wire form may be defined by a coiled spring configured and dimensioned to deploy out of a distal end of the cannula and expand radially, whereby the wire form may be secured and adjusted relative to the anchor by a length of suture or other fiber. In accordance with further embodiments of the present disclosure, the wire form further includes surface roughness or barbs along the interior surface of the wire form to enhance the gripping of tissue. A grasper may be provided that is configured and dimensioned to extend out of the distal end of the cannula and through the wire form, such grasper being effective to grasp tissue and retract it into the wire form.

The disclosed tissue retractor assembly, therefore, while utilizing known medical technologies and current laparoscopic techniques, advantageously provides an anchor which may be used to transfer a force that is delivered from a position external to the abdominal cavity. The force may be delivered through manipulation of a length of suture or fiber relative to the anchor, thereby allowing the surgeon/user to tension or otherwise manipulate a grasper positioned within the abdominal cavity. Indeed, according to the present disclosure, a surgeon may manipulate a grasper relative to an anchor point established in the peritoneal wall or other location within the abdominal cavity.

In accordance with embodiments of the present disclosure, an exemplary tissue retractor assembly is disclosed which may function to deploy a suture around an organ or other anatomical structure, e.g., in a looping manner. The tissue retractor assembly may further be adapted to (i) fire an anchor with an angled sharp tip into an abdominal wall, and (ii) manage a suture that is attached to the loop of suture and routed through the anchor, thus allowing the organ/structure to be retracted by tensioning the suture, e.g., through a port that passes through the abdominal wall. Specifically, the exemplary tissue retractor assembly includes a cannula which houses an anchor and a grasper. The grasper is defined by a loop of suture with a one way locking toggle that is configured and dimensioned to be released/advanced distally from the cannula, the loop of suture being configured and dimensioned to grasp an organ or other anatomical structure, and to retract and tighten around the organ/structure.

In accordance with further embodiments of the present disclosure, the loop suture may include small cuts or barbs to increase the friction of the loop of suture relative to the organ, thereby reducing the possibility of slippage therebetween. The one way locking toggle may be defined by a molded plastic part which allows the loop of suture to be pulled through in one direction, but stops the loop of suture from loosening. Additionally, the anchor may be defined by a substantially symmetrical structure.

The anchor may be defined by a back span, torsion springs and an axial connection between the back span and torsion springs. The anchor may further include two sharpened legs configured and dimensioned to deploy from a distal end of the cannula. The grasper may be adjusted and/or manipulated relative to the anchor by a length of suture that passes therethrough. The disclosed tissue retractor assembly, therefore, while utilizing known medical technologies and current laparoscopic techniques, provides a spring clip anchor which allows penetration of the abdominal tissue with a reduced chance for clinical injury and may be used to transfer a force from a location external to the abdominal cavity. A length of suture or fiber may be used to enable tensioning of the organ grasper, such suture/fiber passing through the anchored point and ultimately passing through the abdominal wall, e.g., through an access port. The disclosed delivery system may advantageously facilitate introduction through a 5 mm port, and permit interaction with both the grasper and the anchor to achieve the clinical results described herein. The disclosed system also generally facilitates management and routing of the suture from the delivery port and permits/facilitates removal of the clip from the abdominal wall.

In accordance with embodiments of the present disclosure, an exemplary tissue retractor assembly is disclosed which fires sequentially deployed 5 mm Raney clips. The Raney clips may be adapted to exit the end of the cannula and are therefore fired axially as opposed to the traditional transverse method. The Raney clip may be atraumatically applied to an organ or structure according to the present disclosure and a second clip may be applied which functions as an anchor in the abdominal wall. A suture may be attached to a grasper and routed through the second clip/anchor, thus allowing the organ/structure to be retracted by tensioning the suture, e.g., from an external location based on the suture passing through the abdominal wall, e.g., through a port.

Specifically, in exemplary embodiments, the tissue retractor assembly may include a cannula which houses a first grasper and a second grasper. The first grasper is defined by a first clip configured and dimensioned to be deployed axially from a distal end of the cannula, and is further defined by a C-shaped form after deployment from the cannula. The second grasper is defined by a second clip configured and dimensioned to be deployed axially from a distal end of the cannula, and is further defined by a C-shaped form after deployment from the cannula. The first grasper is advantageously adapted to be secured and adjusted relative to the second grasper by a length of suture.

In accordance with further embodiments of the present disclosure, the first clip and second clip are metal, plastic or a combination of metal and plastic. The first clip and second clip are further defined by teeth at an open tip or on an inside surface of the first clip and second clip to aid in gripping tissue. The first clip and second slip may further include rubber coating to aid in atraumatically grasping tissue.

The disclosed tissue retractor assembly, therefore, provides both organ and anchor attachment with the same type of clip and deployment technique. The disclosed retractor assembly thus allows the possibility of deployment of multiple clips if advantageous to the procedure, an anchor which may be used to transfer a force, a length of suture or fiber to enable tensioning from the organ grasper through the anchored point, and a delivery system to enable introduction through a 5 mm port. The disclosed system permits attachment/securement of both the grasper and the anchor within the abdominal cavity and permits the surgeon/user to manage/manipulate the grasper through interaction with a suture that passes through the abdominal wall, e.g., through a delivery port. Moreover, the disclosed tissue retractor assembly permits removal of the clip/anchor from the abdominal wall on an as-desired basis.

Additional features, functions and benefits of the disclosed tissue retractor assemblies will be apparent from the detailed description which follows, particularly when read in conjunction with the appended figures.

### BRIEF DESCRIPTION OF FIGURES

To assist those of skill in the art in making and using the disclosed tissue retractor assemblies, reference is made to the accompanying figures wherein:

FIG. 1 is a perspective view of an exemplary tissue retractor cannula housing a grasper and anchor.

FIGS. 2A-C are perspective views of an exemplary tissue retractor at progressive stages of grasping a tissue or organ.

FIG. 3 is a perspective view of an exemplary grasper in an open position.

FIG. 4 is a side view of an exemplary grasper in an open position.

FIG. 5 is a side view of an exemplary grasper in an initial closed position.

FIG. 6 is a side view of an exemplary grasper in a final closed position.

FIG. 7 is a perspective view of an exemplary anchor in a released position.

FIG. 8 is a perspective view of an exemplary anchor with a different configuration in a released position.

FIG. 9 is a perspective view of an exemplary grasper and anchor in operation.

FIG. 10 is a perspective view of an exemplary tissue retractor cannula housing an anchor, a wire form and a grasper.

FIGS. 11A-C are perspective views of an exemplary tissue retractor at progressive stages of deploying an anchor.

FIGS. 12A-E are perspective views of an exemplary tissue retractor at progressive stages of deploying a wire form.

FIG. 13 is a perspective view of an exemplary anchor and wire form in operation.

FIG. 14 is a perspective view of an exemplary tissue retractor cannula housing an anchor and a grasper.

FIG. 15 is a partial section view of an exemplary tissue retractor cannula housing an anchor and a grasper.

FIGS. 16A-D are perspective views of an exemplary tissue retractor at progressive stages of grasping an organ or tissue.

FIGS. 17A-D are perspective views of an exemplary tissue retractor at progressive stages of deploying an anchor.

FIG. 18 is a perspective view of an exemplary anchor and grasper in operation.

FIG. 19 is a partial section view of an exemplary tissue retractor cannula housing a first grasper and a second grasper.

FIGS. 20A-E are perspective views of an exemplary tissue retractor at progressive stages of deploying the first grasper and second grasper.

FIG. 21 is a perspective view of an exemplary first grasper and second grasper in operation.

### DESCRIPTION OF EXEMPLARY EMBODIMENT(S)

In accordance with embodiments of the present disclosure, tissue retractor assemblies are disclosed that generally involve tissue retractors for use in minimally invasive procedures, e.g., in procedures where abdominal access is gained through a single port or multiple ports. Specifically, the tissue retractor assemblies are laparoscopic surgical assist devices which facilitate the retraction of various organs or tissues intracorporeally. The tissue retractor assemblies generally take the form of multi-component devices configured and dimensioned to be delivered through an abdominal wall, e.g., through a 5 mm laparoscopic port, and to provide an atraumatic means to grasp and hold an organ or other anatomical tissue/structure, e.g., the gall bladder. The dimensional characteristics of the disclosed tissue retractor assemblies/systems are generally adapted for use through a 5 mm cannula commonly encountered in the use and operation of laparoscopic surgical tools.

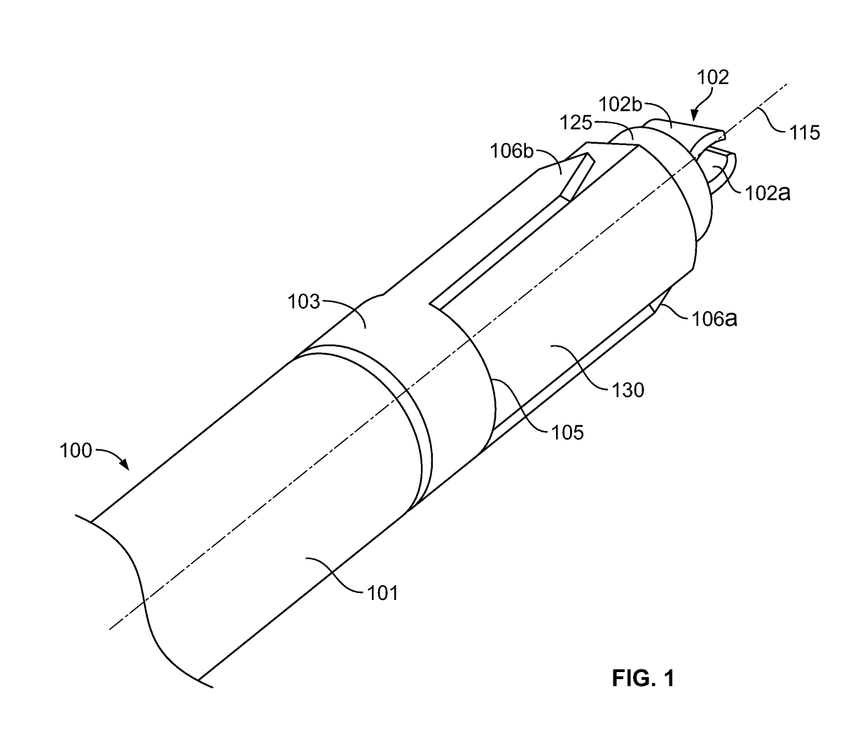

With reference to FIG. 1, an exemplary embodiment of a tissue retractor assembly is depicted in accordance with the present disclosure in the form of a tissue retractor assembly 100. The tissue retractor assembly 100 includes a cannula 101 which houses a grasper 102 and an anchor 103. The grasper 102 is configured and dimensioned to be extended axially from a distal end of the cannula 101 and includes a first leg 102a and a second leg 102b for gripping tissue. The first leg 102a and second leg 102b may be fabricated from flat sheet metal or plastic. Further, first and second leg 102a and 102b may optionally be coated with a rubber and may have surface features or a shape that is/are advantageous to grasping without damaging the organ. The grasper 102 further comprises a locking ring 104 which is configured and dimensioned to be pushed distally by a pusher cannula 125 to grasp and lock the first leg 102a and second leg 102b around an organ or tissue.

The tissue retractor assembly 100 further includes the anchor 103 which is configured and dimensioned to be deployed from the cannula 101 and attached to an abdominal wall anterior to an organ. The anchor 103 is further defined by a cylindrical body 105 attached to at least two sharpened thin legs, 106a and 106b, respectively, that lie along an axis 115 of the cylindrical body 103. The at least two sharpened thin legs 106a and 106b are preformed into a preformed shape that when the anchor 103 is deployed by the cannula 101, the at least two sharpened thin legs 106a and 106b return to the preformed shape to increase a pull out force of the anchor 103. As shown in FIG. 1, legs 106a and 106b may rest on an exterior surface of cannula 130. Cannula 130 may advantageously define a “D-shape” such that the relatively flattened sides of cannula 130 effective interact with and support legs 106a, 106b until deployment thereof.

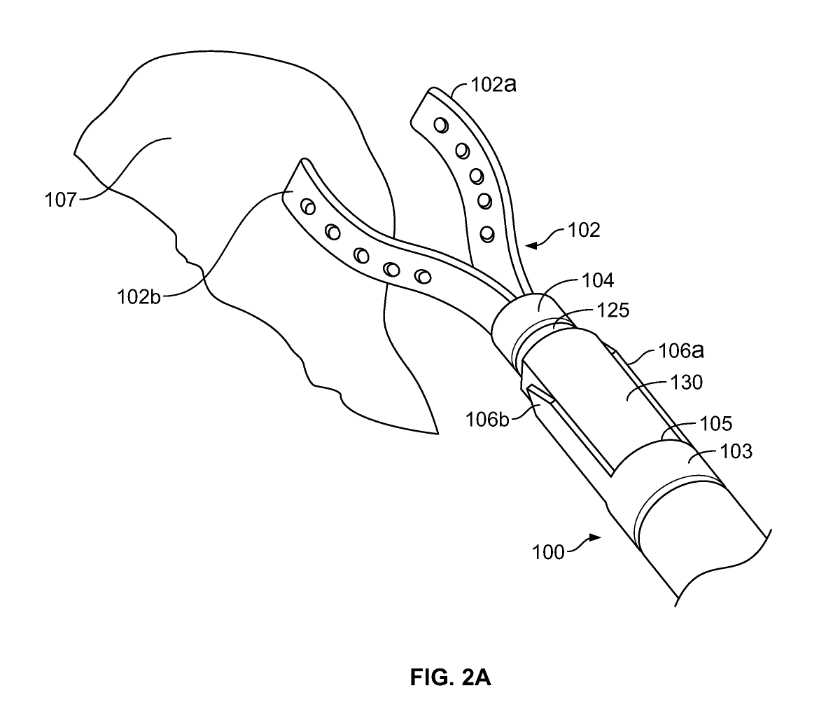

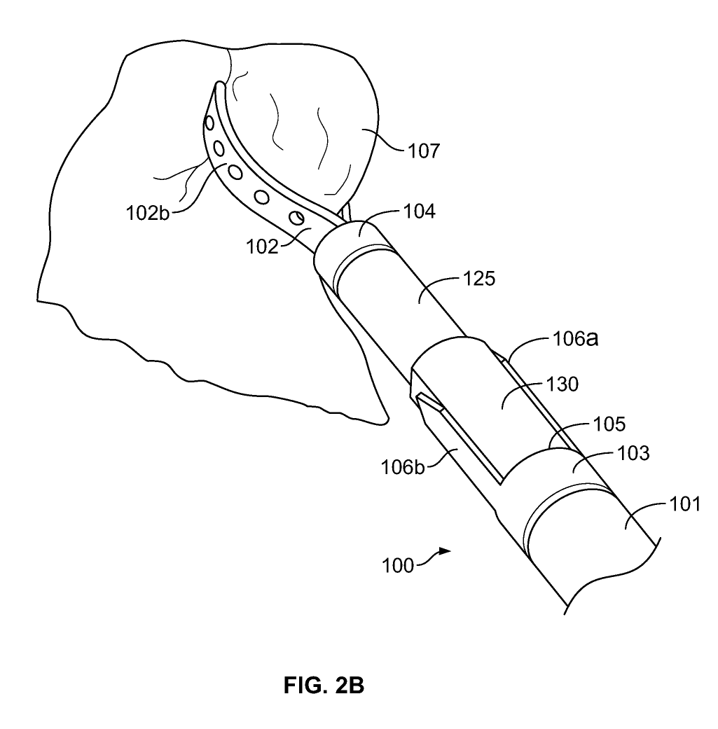

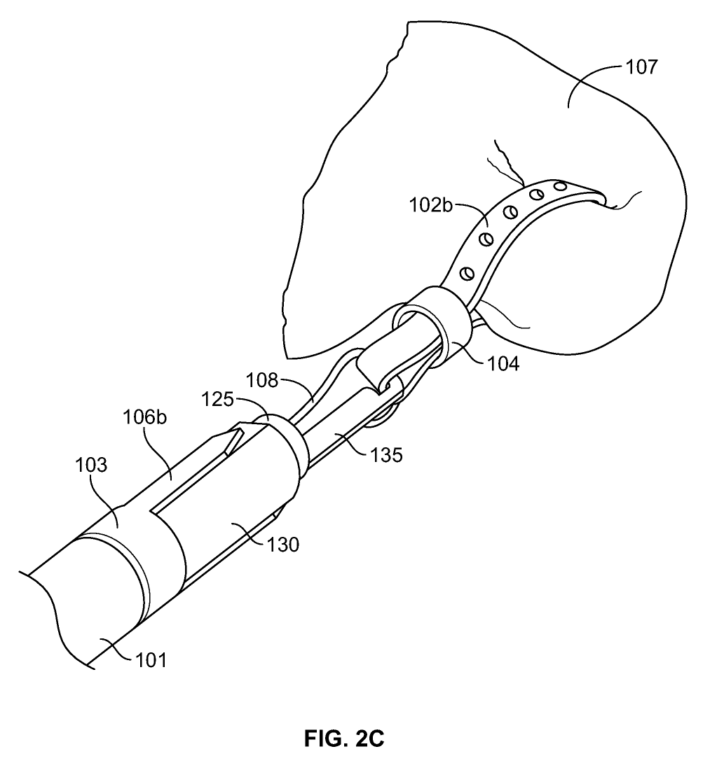

With reference to FIGS. 2A-B, the tissue retractor assembly 100 is depicted at progressive steps of grasping an organ or tissue 107 after the tissue retractor assembly 100 has been introduced into an access port (not shown). With specific reference to FIG. 2A, the tissue retractor assembly 100 is depicted with the grasper 102, which had been loaded into the cannula 101 for purposes of introduction into the port, and the first leg 102a and second leg 102b have been extended out of a distal end of the cannula 101. Once inside the port, and at the organ 107 to be grasped, the grasper 102 is pushed out of the distal end of the cannula 101, e.g., by a hook member 135 attached to the proximal end of the grasper 102. Generally, the clinician has a multipurpose 5 mm grasper in the surgical field during the procedure for managing the tissue of the organ in question. As depicted in FIG. 2A, the first leg 102a and the second leg 102b of the grasper 102 have been extended from the distal end of the cannula 101 and are used to surround the organ 107 to be grasped.

With reference to FIG. 2B, the grasper 102 has been positioned sufficiently around the organ 107 to be grasped and the locking ring 104 is utilized to lock the grasper 102 around the organ 107. Specifically, the locking ring 104 is pushed distally by the pusher cannula 125 while the interior hook member 135 holds the grasper 102 in position. Thus, the locking ring 104 clenches the first leg 102a and second leg 102b around the organ 107.

With reference to FIG. 2C, once the grasper 102 is locked, the cannula 101 is retracted and lifted (or otherwise manipulated) to release the hook member 135. As depicted in FIG. 2C, the tissue retractor assembly 100 further includes a suture 108 which secures and allows adjustment of the grasper 102 relative to the anchor 103. Specifically, the suture 108 is looped relative to the apex of the first and second legs 102a and 102b of grasper 102, and the cannula 101 trails the suture from the distal end thereof.

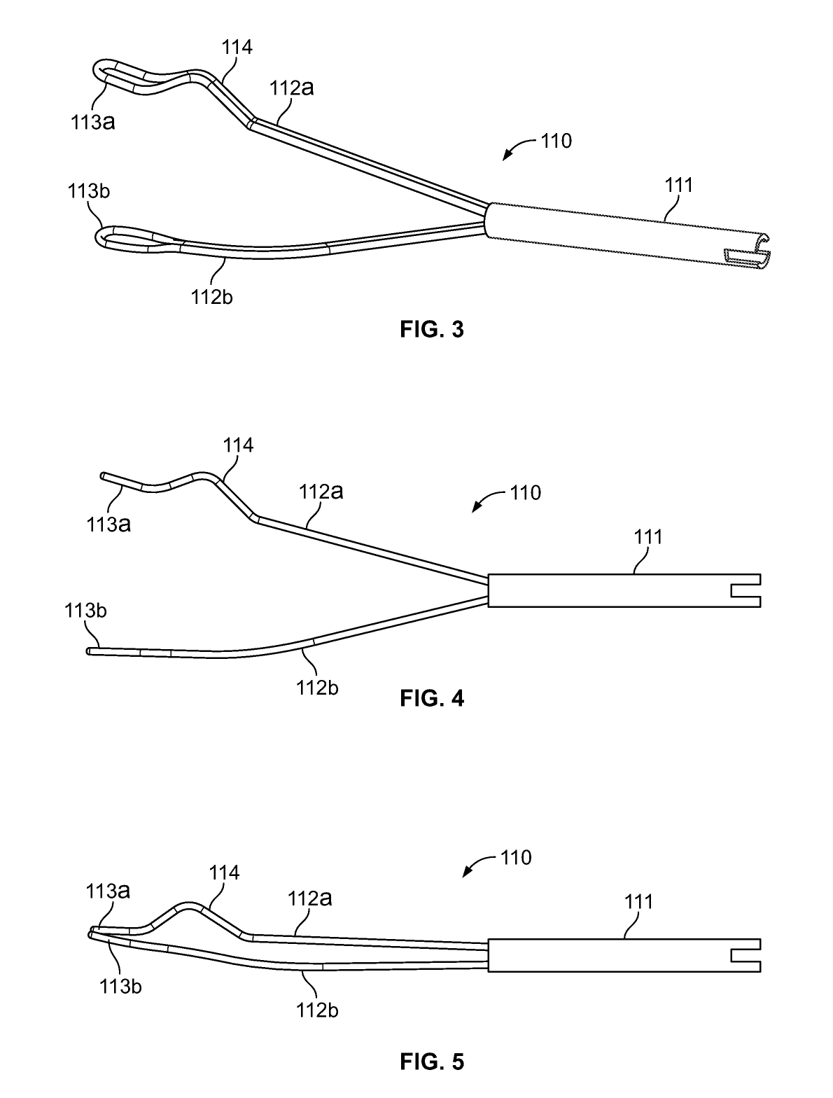

Turning now to FIGS. 3-6, an alternate grasper 110 is depicted for providing a stronger force for gripping an organ or tissue. With reference to FIG. 3, the grasper 110 is depicted in an “open” position and includes a first leg 112a and second leg 112b and a locking sleeve 111. The first and second leg 112a and 112b may be made of stainless wire and may be formed into shape. Additionally, at least one of the first and second legs 112a and 112b may have surface features, e.g., serrations, on a distal end of the first leg 113a and/or a distal end of the second leg 113b. The first leg 112a additionally may include a tissue capture area 114 between the distal end of the first leg 113a and the distal end of the locking sleeve 111. Specifically, the tissue capture area 114 includes angled bends up and down relative to the surface of the first leg 112a, wherein the angled bends may be approximately 45° and thus provide a space for capturing tissue when the first leg 112a and second leg 112b are clinched shut. Further with reference to FIG. 3, the locking sleeve 111 is depicted and may be formed from a long tube.

With reference to FIG. 4, an additional side view of the alternate grasper 110 in an “open” position is depicted for providing a clearer view of the tissue capture area 114. The second leg 112b may be either formed in a straight manner or may contain a curve in order to provide a stronger grip on the organ or tissue when the first leg 112a and second leg 112b are clinched shut.

With reference to FIG. 5, the grasper 110 is depicted in a “closed” position. As the locking sleeve 111 is pushed distally by a pusher cannula, e.g., pusher cannula 125, in the direction of the distal end of the first and second leg 113a, 113b, the first leg 112a and second leg 112b clinch shut. Thus, the clinching of first and second legs 112a, 112b is achieved by relative axial motion of the pusher cannula and the legs of the grasper, e.g., the pusher cannula moves distally while the grasper legs are maintained in a fixed axial position. Alternatively, the grasper legs could be moved proximally relative to a fixed cannula, but such relative motion would cause proximal movement of the grasper legs relative to the tissue/organ of interest which is likely undesirable in clinical use.

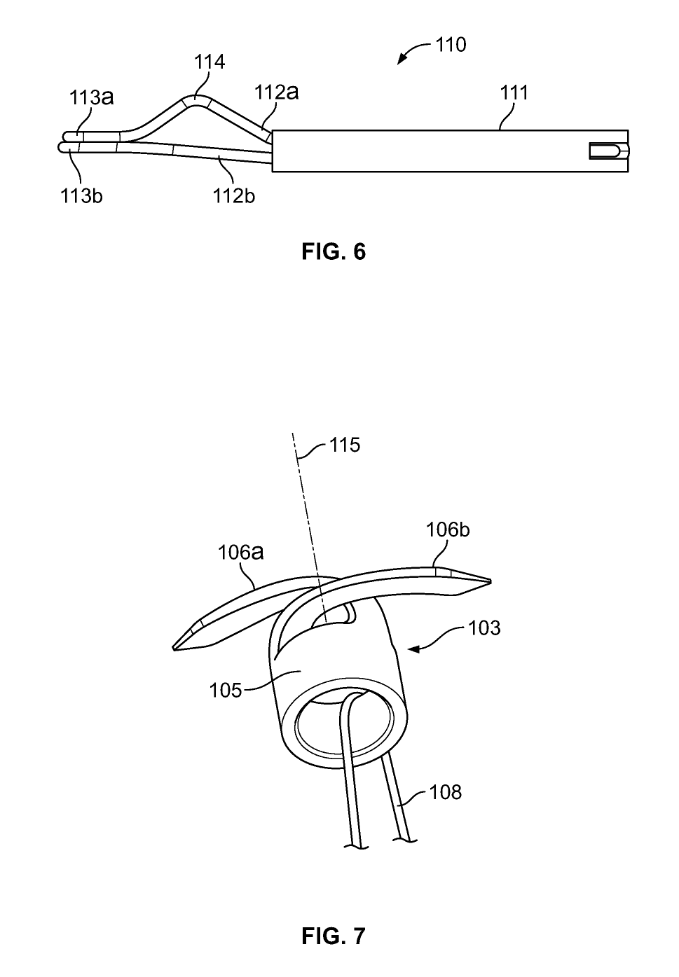

With reference to FIG. 6, the grasper 110 is depicted in a “closed” position where the locking sleeve 111 has been pushed distally by a pusher cannula, e.g., pusher cannula 125, to an appropriate point of closure, i.e., to achieve a desired grasping force. It should be understood by those with ordinary skill in the art that as the locking sleeve 111 is advanced distally relative to first and second legs 113a and 113b, the first leg 112a and second leg 112b would be pushed together with an increasing force, thereby providing the clinician a range of forces which may be applied by the grasper 110 in order to sufficiently grip the organ or tissue.

Turning now to FIG. 7, an exemplary embodiment of the anchor 103 is depicted. The tissue retractor assembly 100 is used to deploy the anchor 103 to an abdominal wall. The anchor 103 may be fabricated from a preformed shape memory nitinol staple which could be formed from a single wire form or cut from a tube. The fundamental structure of the anchor 103 is a cylindrical body 105 attached to at least two sharpened thin legs 106a and 106b that lie along an axis 115 of the cylindrical body 105. The at least two sharpened thin legs 106a and 106b are sharpened to allow them to penetrate tissue. The preformed nature of the at least two sharpened thin legs 106a and 106b allows the at least two sharpened thin legs 106a and 106b to return to their preformed shape in order to increase the pull out force of the anchor. As depicted in FIG. 1, the anchor 103 is initially loaded over a “D” or specifically shaped cannula 101 that helps to constrain the at least two sharpened thin legs 106a and 106b and allows the anchor 103 to be deployed by a simple tube over a tube push mechanism. With reference to FIG. 7, the anchor 103 is depicted in a “released” position, wherein the at least two sharpened thin legs 106a and 106b were preformed to fold in towards the axis 115 and center of the cylindrical body 106.

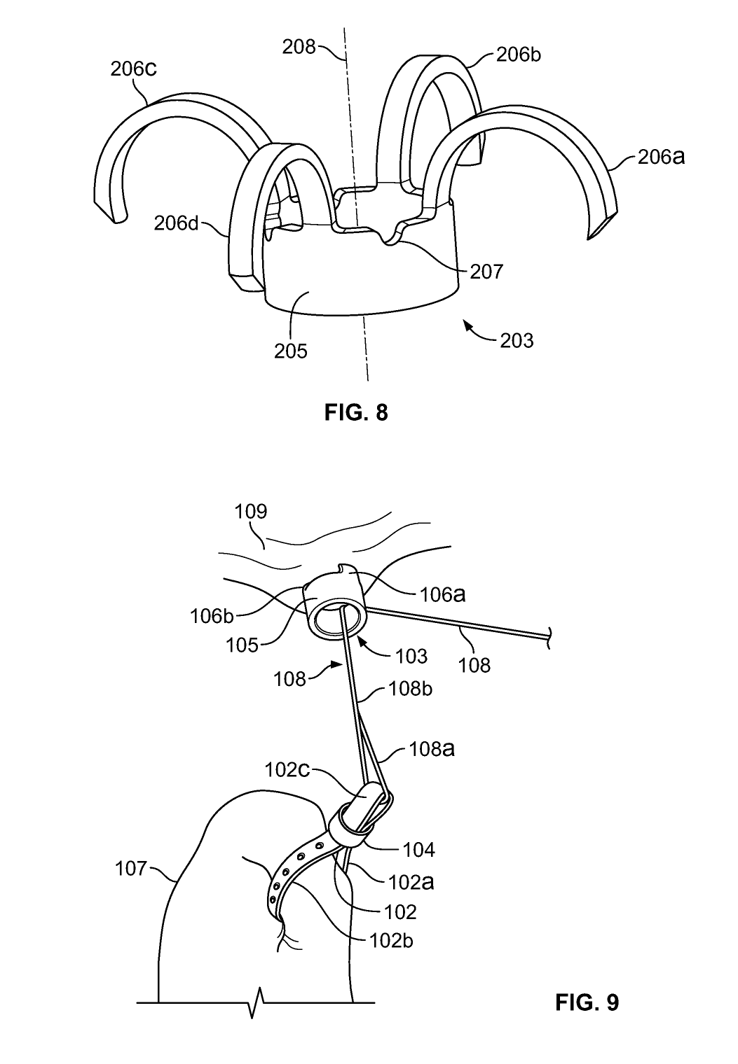

With reference to FIG. 8, an alternate exemplary embodiment of the anchor 203 is depicted. Unlike the anchor 103 in FIG. 7, the anchor 203 in FIG. 8 includes the at least two sharpened thin legs 206a, 206b, 206c and 206d which were preformed to fold away from the axis 208 and the center of the cylindrical body 205. As in FIG. 7, the anchor 203 of FIG. 8 also has cylindrical body 205 as the fundamental structure of the anchor 203, and the cylindrical body 205 is attached to the at least two sharpened thin legs 206a, 206b, 206c and 206d. The anchor 203 may also include grooves 207 in the cylindrical body 205 for purposes of guiding the suture 108.

With reference to FIG. 9, the exemplary anchor 103 or 203 is attached by positioning the distal tip of the D-shaped cannula 130 on the abdominal wall 109, the clinician's hand usually palpates on the outside of the abdominal wall 109, the cannula 101 pushes the anchor 103 or 203 axially distal off of the D-shaped cannula 130 as the anchor 103 or 203 penetrates the tissue 109. As the anchor 103 or 203 is released from the D-shaped cannula 130, the at least two sharpened thin legs 106a and 106b or 206a, 206b, 206c and 206d curve in or away from the center of the cylindrical body 105 or 205 and thus provide a sufficient pull out force as well as protect the clinician from injury.

FIG. 9 further depicts the exemplary grasper 102 and anchor 103 in operation. Specifically, the grasper 102 has clinched the organ 107 and has been locked in position by the locking mechanism 104. Additionally, the anchor 103 has been released from the cannula 101 and the preformed at least two sharpened thin legs 106a and 106b have returned to their preformed shape in order to provide a sufficient pull out force. It should be noted that the grasper 102 is movably secured to the anchor 103 by the suture 108. The cannula 101 is retracted from the port trailing the suture 108 which keeps all the components tethered and allows the clinician to retract the organ 107 by increasing the tension on the suture 108. As shown therein, grasper 102 is fixed with respect to tissue/organ 107 with suture 108 looped through a U-shaped extension region 102c thereof. In the exemplary implementation of FIG. 9, suture 108 defines a loop region 108a that interacts with U-shaped extension region 102c of grasper 102, such loop region extending to suture junction 108b. A single suture strand 108 extends from suture junction 108b and extends through anchor 103 which is fixed relative to a second tissue location 107a, e.g., a peritoneal wall of the patient. The legs of anchor 103 are positioned within second tissue location 107a, e.g., in a crossing orientation. The free end of suture 108 generally extends through the abdominal wall, e.g., through an access port (not pictured), and permits manipulation of tissue/organ 107 relative to anchor 103. The deployment tool has been withdrawn from the surgical field. The suture 108 can be secured outside the port with a clamp or other appropriate means. At the end of the surgery, the grasper 102 will be removed with the organ 107 (in the case of a gall bladder removal). The anchor 103 can be removed by gripping it with a 5 mm grasper (not shown) and pulling along the axis of the anchor 103 to remove it from the abdominal wall. Both parts of the tissue retractor assembly 100 can be removed through the abdominal incision created by the introduction of the port.

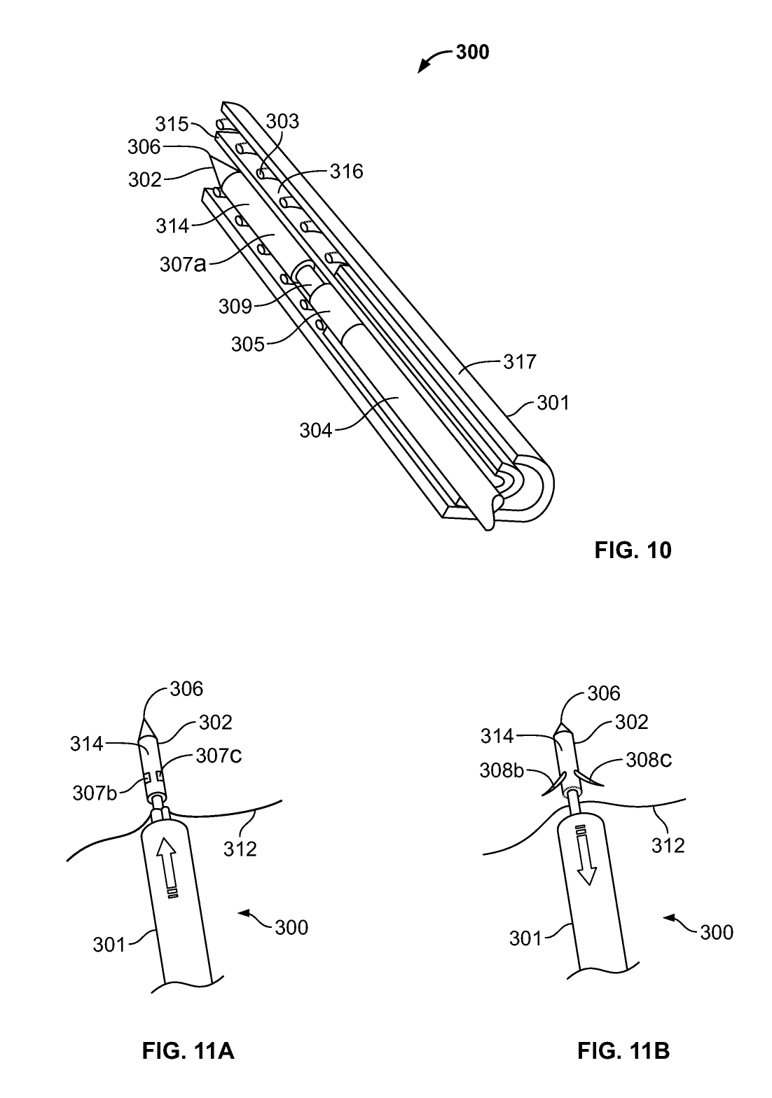

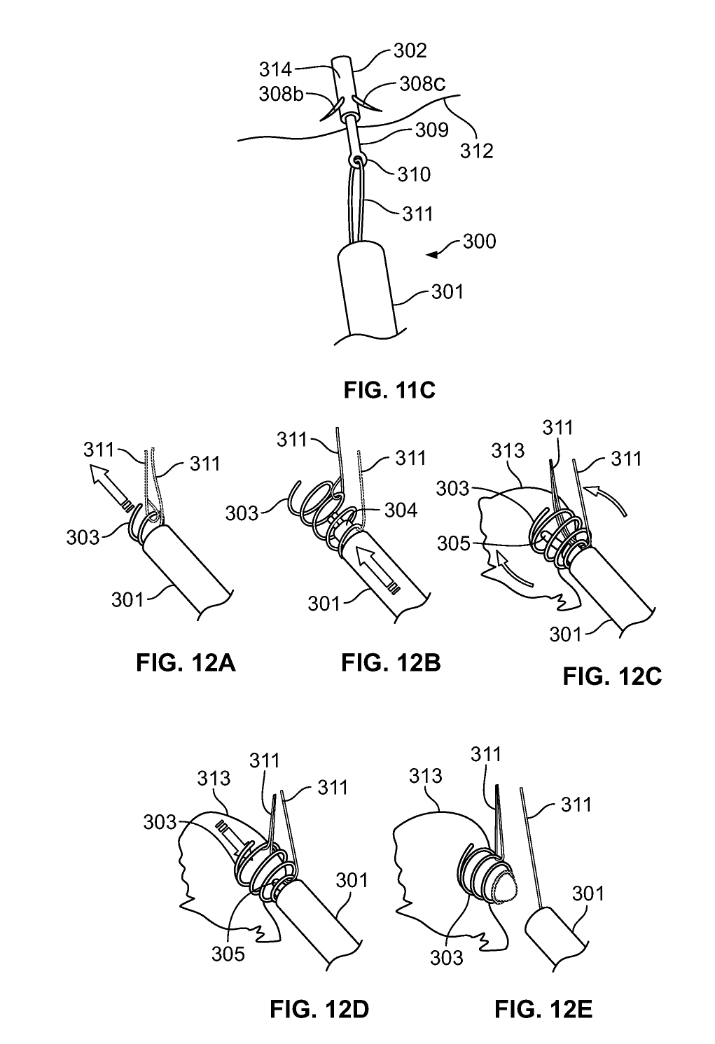

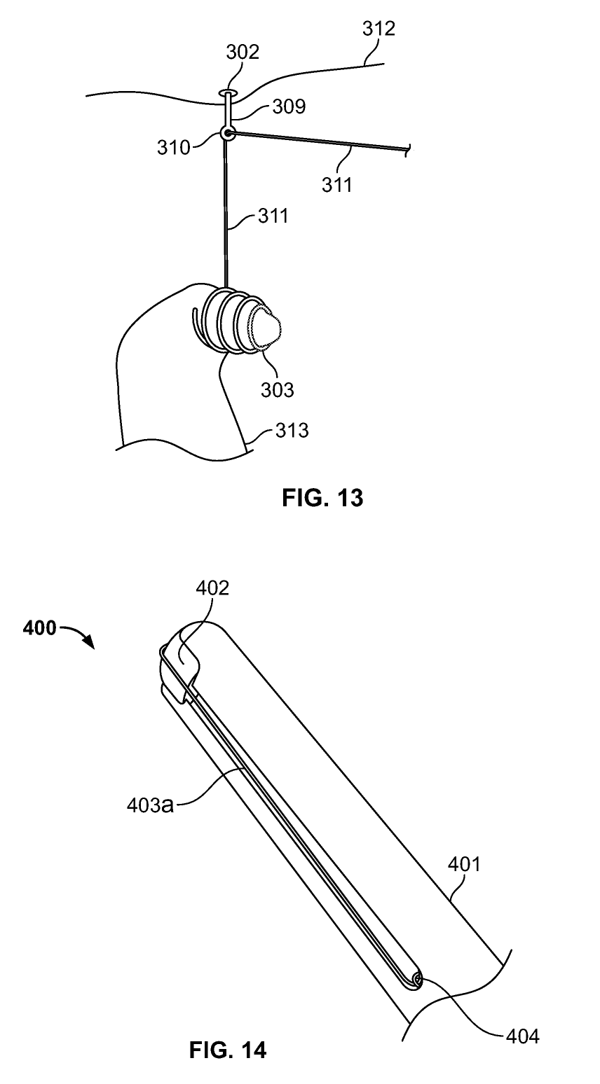

Now turning to FIG. 10, an alternate tissue retractor assembly 300 is depicted in accordance with the present disclosure. In the exemplary embodiment of FIG. 10, the tissue retractor assembly 300 includes a cannula 301 which houses an anchor 302, a wire form 303 and a grasper 304. The anchor 302 is configured and dimensioned to be deployed from the cannula 301 and attached to an abdominal wall anterior to an organ. The anchor 302 includes an outer tube 314 and a central shaft 309, the central shaft 309 further including at least two barbs 308a and 308b, depicted in FIG. 11B, which are configured to be deployed from the outer tube 314 when the central shaft 309 is axially pulled. Additionally, the anchor 302 includes a retractable sharp tip 306 which retracts into the outer tube 314 when the central shaft 309 is pulled axially to deploy the at least two barbs 308a and 308b. The wire form 303 may be fabricated as a coiled spring configured and dimensioned to deploy out of a distal end of the cannula 301 and expand radially. Further, the wire form 303 is secured and adjusted relative to the anchor 302 by a length of suture 311. Lastly, the grasper 304 is configured and dimensioned to extend out of the distal end of the cannula 301 and through the wire form 303, grasp tissue 313 (depicted in FIG. 13), and retract into the distal end of the cannula 301 to pull tissue 313 into the wire form 303. The grasper 304 may be a pediatric-type grasper with specialized jaws 305.

Still with reference to FIG. 10, the tissue retractor assembly 300 is based upon a 5 mm cannula 301 commonly used in the design of laparoscopic surgical tools. The cannula 301 contains both the anchor 302 and the wire form 303 used to grasp the organ or tissue 313. The components of the cannula 301 are arranged coaxially with the anchor 302 in the center with a fully functional 3 mm grasper 304 proximal to the anchor 302. Separated by a cannula wall 315, the wire form 303 is compressed into an annular ring 316 surrounded by the outer cannula wall 317.

The anchor 302 is constructed in two pieces, the outer tube 314 which forms the body of the anchor 302 and internal to the outer tube 314, the central shaft 309 which includes the retractable sharp tip 306 and at least two barbs 308a and 308b integrated that can be deployed by pulling the central shaft 309 of the anchor 302 proximal to the retractable sharp tip 306. The anchor 302 may be fabricated from metal or plastic.

With reference to FIGS. 11A-C, the tissue retractor assembly 300 is depicted at progressive steps of securing the anchor 302 to the abdominal wall 312 after the tissue retractor assembly 300 has been introduced into an access port (not shown). Initially, the distal tip of the cannula 301 is positioned on the abdominal wall 312. The clinician's hand usually palpates on the outside of the abdominal wall 312. A specially designed 3 mm grasper 304, commonly used in pediatric laparoscopic procedures, is utilized to push the anchor 302 axially distal to penetrate the tissue 312. The 3 mm grasper 304 then retracts in order to retract the sharp tip 306 and deploy the at least two barbs 308a and 308b, as depicted in FIGS. 11A and 11B. Specifically, the at least two barbs 308a and 308b deploy through openings 307a and 307b in the outer tube 314 of the anchor 302. The at least two barbs 308a and 308b dramatically increase the holding force of the anchor 302 in the abdominal wall 312. As depicted in FIG. 11C, the anchor 302 further has a suture 311 attached to the proximal end and the cannula 301 trails the suture 311 from the distal tip. The suture 311 may be attached with respect to the proximal end of the central shaft 309 of the anchor 302 by a ring 310 or similarly shaped component.

With reference to FIGS. 12A-E, the tissue retractor assembly 300 is depicted at progressive steps of grasping an organ 313 after the tissue retractor assembly 300 has been introduced into the port and the anchor 302 has secured to the abdominal wall 312. Generally, a clinician has a multipurpose use 5 mm grasper (not shown) in the surgical field during the procedure. The 5 mm grasper manages the tissue of the organ in question during the grasping of the organ. The wire form 303 is in essence a specifically designed coiled spring which may have features such as surface roughness or barbs along the interior wire surface to enhance the gripping of the organ 313. As depicted in FIG. 12A, the wire form 303 is pushed out of the distal end of the cannula 301 and expands radially to enable a larger diameter profile which can accommodate more tissue of the organ 313. The 3 mm grasper 304 is then extended from the distal end of the cannula 301 to reach through the wire form 303, grasp the organ 313 and retract back into the distal end of the cannula 301 to pull tissue of the organ 313 into the wire form 303, which will grip the organ 313 by virtue of the forces generated between the surfaces of the wire form 303 and organ 313. With reference to FIG. 12E, once the wire form 313 has been secured around the organ 313, the wire form 303 is secured and adjusted relative to the anchor 302 by a length of suture 311. Specifically, the length of suture 311 is attached to the wire form 303, extends to the ring 310 of the anchor 302, as depicted in FIG. 11C, and is attached to the cannula 301.

With reference to FIG. 13, the exemplary wire form 303 and anchor 302 are depicted in operation. Once the wire form 303 and anchor 302 are attached to the organ 313 and abdominal wall 312, respectively, the cannula 301 is retracted from the access port trailing the length of suture 311, which keeps all the components tethered and allows the clinician to retract the organ 313 by increasing the tension on the length of suture 311. The length of suture 311 can be secured outside the port with a clamp or other appropriate means (not shown). At the end of the surgery, the grasper 303 will be removed with the organ 313 (in the case of a gall bladder removal). Removal of the anchor 302 will require the reintroduction of the cannula 301, which contains the 3 mm grasper 304. A 5 mm grasper could be used to grip the outer tube 314 of the anchor 302, while the 3 mm grasper 304 is used to attach to the central shaft 309 of the anchor 302 and push distally to retract the at least two barbs 308a and 308b to allow the anchor 302 to be removed from the abdominal wall 312. The anchor 302 could be retracted into the cannula 302 or removed through the 5 mm port individually. Both components of the cannula 301 may also be removed through an abdominal incision created by the introduction of the access port as both are tethered to the length of suture 311.

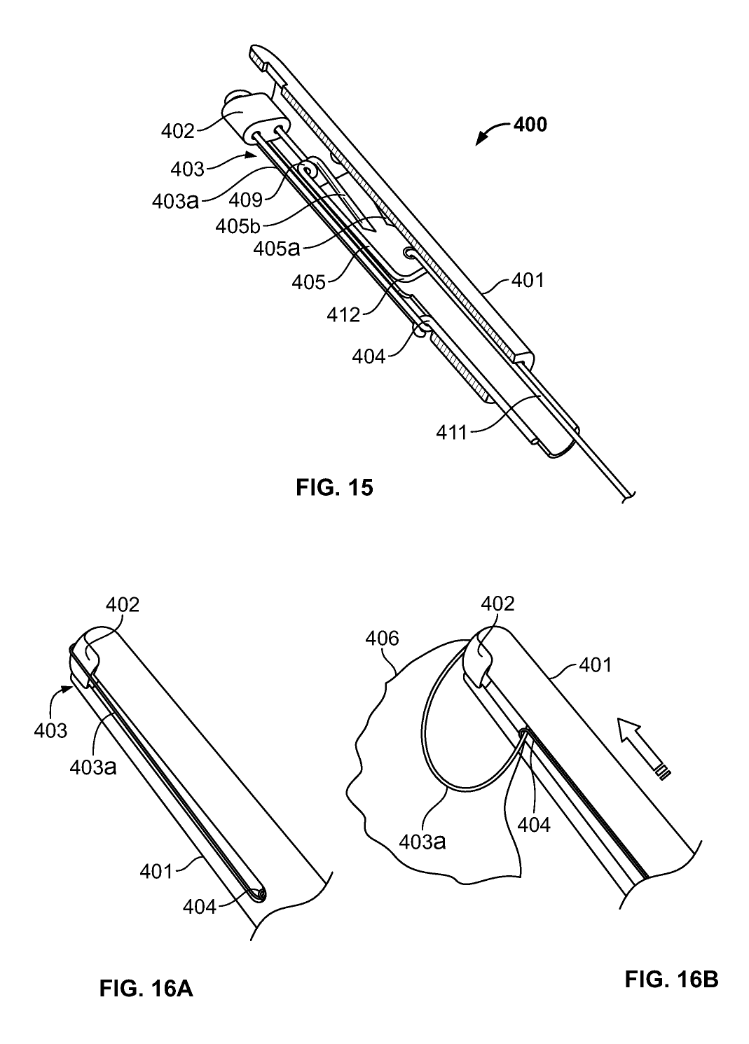

Now turning to FIGS. 14 and 15, an alternate tissue retractor assembly 400 is depicted in accordance with the present disclosure. FIG. 15 provides a partial view of the alternate tissue retractor assembly 400 for a more convenient depiction of the internal components of the tissue retractor assembly 400. In the exemplary embodiment of FIGS. 14 and 15, the tissue retractor assembly 400 includes a cannula 401, which houses an anchor 405 and a grasper 403. The grasper 403 is comprised of a loop of suture 403a with a one way locking toggle 402, configured and dimensioned to be released distally from the cannula 401. Specifically, the loop of suture 403a is configured and dimensioned to grasp tissue of an organ 406, retract into the distal end of the cannula 401 and tighten around the tissue of the organ 406. The anchor 405 includes a back span 412, torsion springs 409 and an axial connection between the back span 412 and torsion springs 409. The anchor 405 further includes two sharpened legs 405a and 405b configured and dimensioned to deploy from a distal end of the cannula 401. The grasper 403 is secured and adjusted relative to the anchor 405 by a length of suture 411.

Further with reference to FIGS. 14 and 15, the tissue retractor assembly 400 is based upon a 5 mm cannula 401 commonly used in the design of laparoscopic surgical tools. The cannula 401 contains both the grasper 403 to attach to an organ 406 and an anchor 405, as well as a system to deploy each. The components of the cannula 401 are arranged with both the grasper 403 and anchor 405 along the axis of the cannula 401 with the grasper 403 below the anchor 405, which may be fabricated as a spring clip. The cannula 401 may further include a slot to allow the deployment of the loop of suture 403a. The cannula 401 further includes features to aid in the delivery and firing or deployment of the grasper 403 and anchor 405.

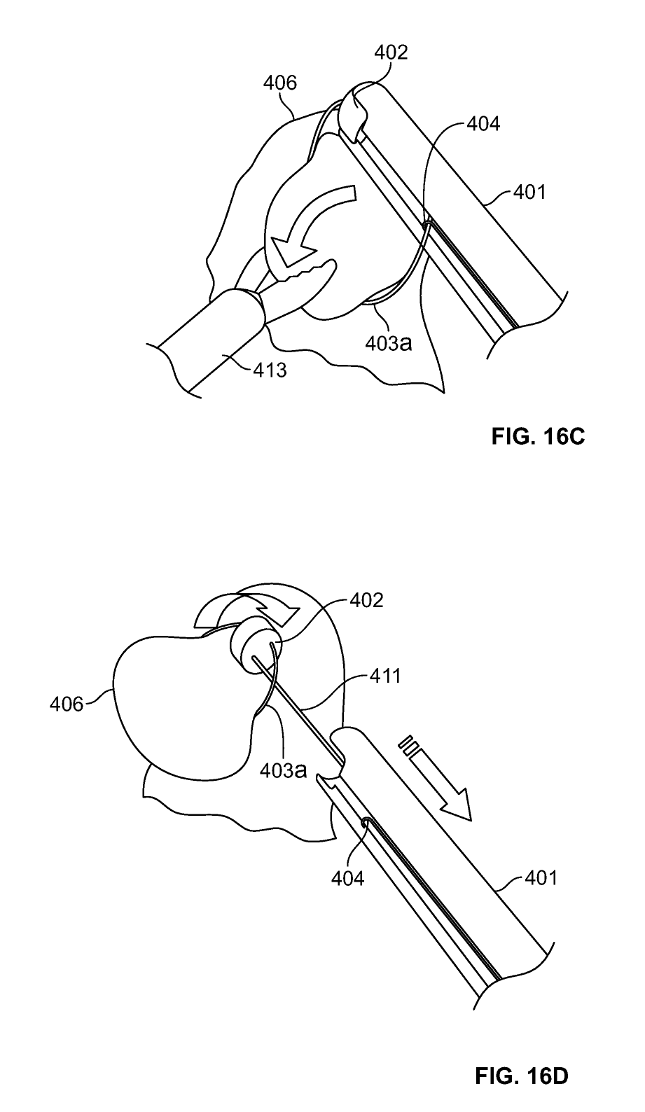

With reference to FIGS. 16A-D, the tissue retractor assembly 400 is depicted at progressive steps of grasping an organ 406 after the tissue retractor assembly 400 has been introduced into an access port (not shown). The grasper 403 is a suture based organ grasper including a loop of suture 403a with a one way locking toggle 402. The one way locking toggle 402 may be fabricated as a small molded plastic part which allows the loop of suture 403a to be pulled through in one direction, but stops the loop of suture 403a from loosening. The loop of suture 403a may also be a ribbon or similar structure to increase friction or distribute force more evenly. Additionally, the loop of suture 403a may have surface features, i.e., small cuts or barbs, on its diameter to increase the friction of the loop of suture 403a to the organ 406 and reduce the possibility of slipping. The loop of suture 403a is held flat in the cannula 401 by a hook 404 which is in the loop of suture 403a and holds tension on the loop of suture 403a in the cannula 401.

Still with reference to FIGS. 16A-D, in order to attach the loop of suture 403a to the organ 406, the cannula 401 would be introduced through an access port and placed near the attachment site. The loop of suture 403a would be moved distal in order to produce slack in the loop of suture 403a. Generally, the clinician has a multipurpose use 5 mm grasper 413 in the surgical field during the procedure. The 5 mm grasper 413 manages the tissue of the organ 406 in question during the grasping of the organ 406. The 5 mm grasper 513 would be used to pull the tissue of the organ 406 through the loop of suture 403a. The delivery portion of the cannula 401 would pull the free end of the loop of suture 403a through the one way locking toggle 402 to tighten the loop of suture 403a around the tissue of the organ 406. By retracting the hook 404 and withdrawing the cannula 401, the one way locking toggle 402 and grasper 403 assembly could be released from the cannula 401. The free end of the loop of suture, a length of suture 411, would be trailed out of the distal end of the cannula 401 while approaching the attachment point for the anchor 405 to be attached to the abdominal wall 410.

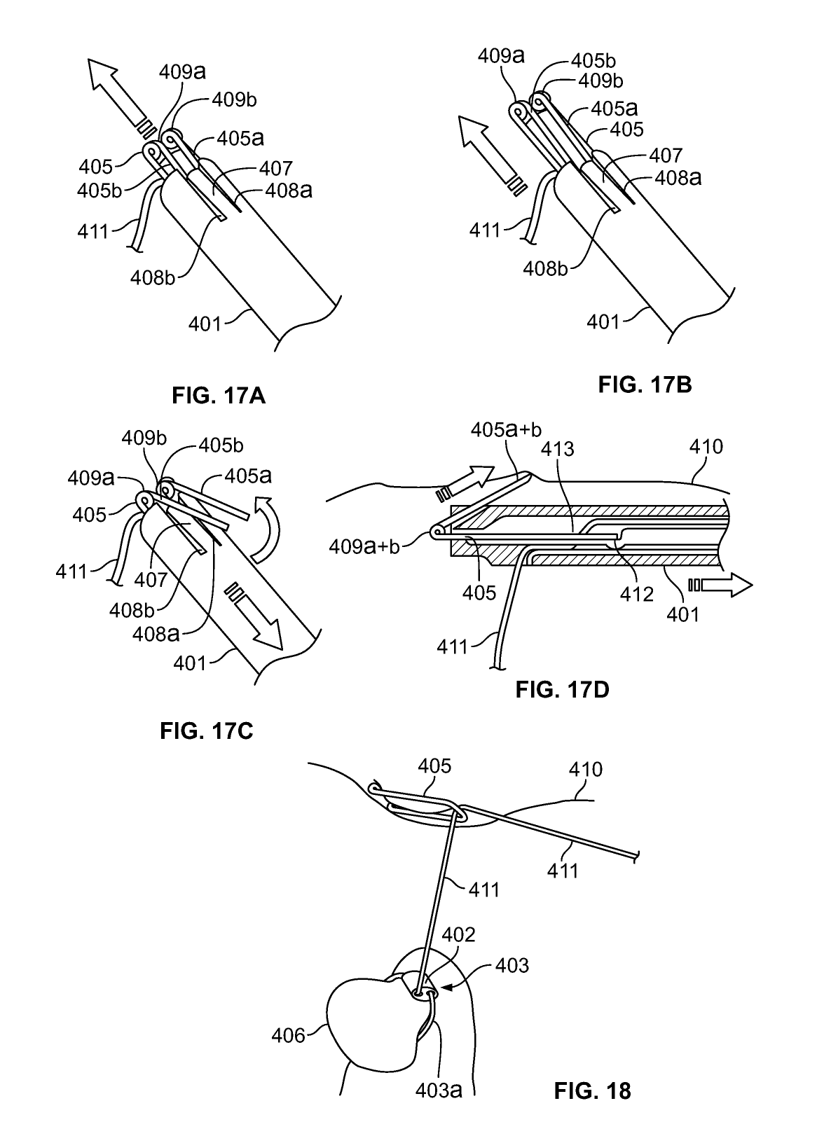

With reference to FIGS. 17A-D, the tissue retractor assembly 400 is depicted at progressive steps of securing the anchor 405 to the abdominal wall 410 after the tissue retractor assembly 400 has been introduced into an access port (not shown) and after the grasper 403 has been secured around the organ 406. The anchor 405 may be fabricated as a wire form constructed from a single piece of wire. The wire is a form which has a substantially symmetrical structure, consisting of a back span 412, torsion springs 409 and axial connections between the elements. The anchor 405 has a structure similar to the normally closed springs used in the typical construction of cloths pins. The anchor 405 further includes two sharpened legs 405a and 405b which are not connected by a cross member and are sharpened to facilitate tissue penetration. The anchor 405 is normally closed and resides in the cannula 401 in a tray 413, or similar structure, for deployment.

Still with reference to FIGS. 17A-D, the deployment of the anchor 405 requires that a device internal to the cannula 401 push the anchor 405 distal enough that the stripping feature 407 on the cannula 401 can wedge under the two sharpened legs 405a and 405b of the anchor 405. The tray 413 is then retracted proximally, which positions the two sharpened legs 405a and 405b of the anchor 405 to penetrate the abdominal wall 410. Specifically, the cannula 401 includes the stripping feature 407 and two slits 408a and 408b, which are dimensioned and configured to allow the two sharpened legs 405a and 405b of the anchor 405 to deploy from the distal end of the cannula 401 by an internal retracting mechanism when the anchor 405 has been partially deployed from the distal end of the cannula 401. Therefore, while the anchor 405 is normally closed in the tray 413, the two sharpened legs 405a and 405b can deploy from the cannula 401 through the two slits 408a and 408b in order to properly penetrate and attach to the abdominal wall 410.

Further with reference to FIGS. 17A-D, the distal tip of the cannula 401 is positioned adjacent to the abdominal wall 410. The clinicians hand usually palpates on the outside of the abdominal wall 410. The clinician would push the cannula 401 anterior, while pulling the cannula 401 and anchor 405 proximally. This would cause the two sharpened legs 405a and 405b of the anchor 405 to snag and penetrate the abdominal wall 410. The anchor 405 would then be released from the cannula 401 by retracting the cannula 401 and pushing the tray 413 distal. The closing action of the anchor 405 and the direction of the tension applied by the length of suture 411 would increase the holding force of the anchor 405.

With reference to FIG. 18, the exemplary anchor 405 and grasper 403 are depicted in operation. The cannula 401 is retracted from the access port trailing the length of suture 311, which keeps all parts tethered and allows the clinician to retract the organ 406 by increasing the tension on the length of suture 411. The length of suture 411 can be secured outside the port with a clamp or other appropriate means. At the end of the surgery, the grasper 403 may be removed with the organ 406 (in the case of a gall bladder removal). The grasper 413, depicted in FIG. 16C, may further be used to grip the back span 412 of the anchor 405 and push away from the entry direction, thereby allowing the anchor 405 to be easily removed. The normally closed nature of the anchor 405 would render the two sharpened legs 405a and 405b safe in the abdominal cavity. Both the anchor 405 and grasper 403 of the tissue retractor assembly 400 may also be removed through the abdominal incision created by the introduction of the access port as both are tethered to the suture.

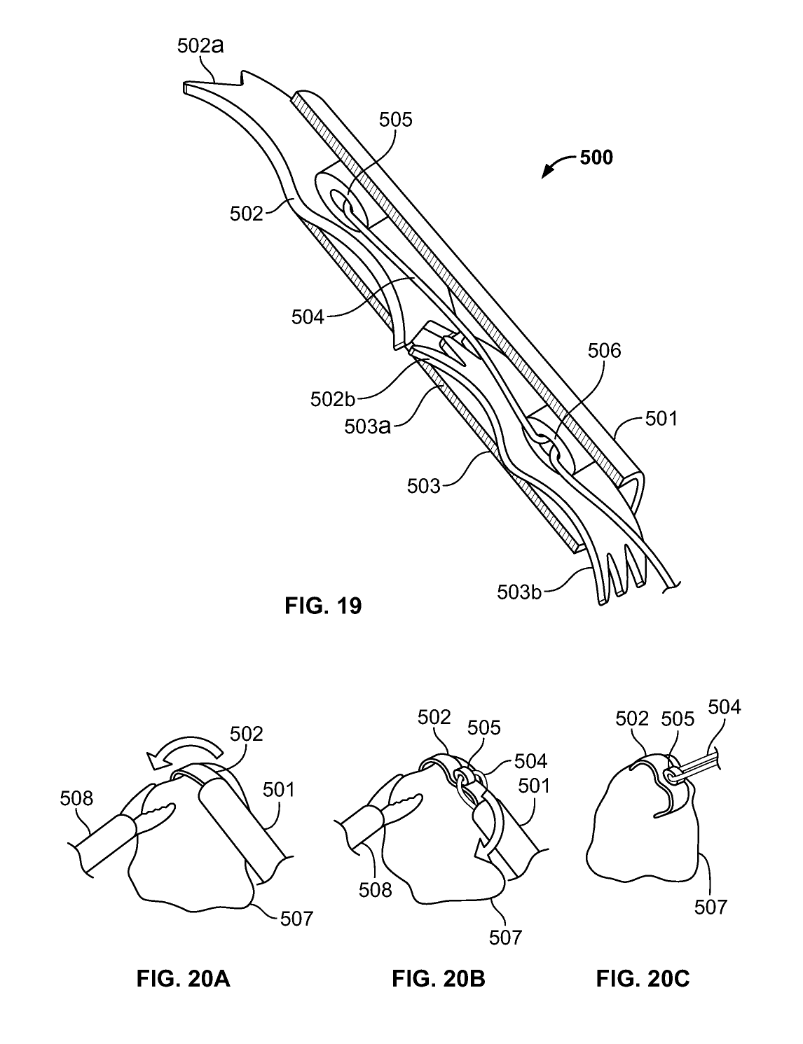

Now turning to FIG. 19, an alternate tissue retractor assembly 500 is depicted in accordance with the present disclosure. In the exemplary embodiment of FIG. 19, the tissue retractor assembly 500 includes a cannula 501, which houses a first grasper 502 and second grasper 503. The first grasper 502 may be fabricated as a first clip configured and dimensioned to be deployed axially from a distal end of the cannula 501 and is defined by a C-shaped form after deployment from the cannula 501. The second grasper 503 may be fabricated as a second clip configured and dimensioned to be deployed axially from the distal end of the cannula 501 and is also defined by a C-shaped from after deployment from the cannula 501. The first grasper 502 is further secured and adjusted relative to the second grasper 503 by a length of suture 504, which is pre-threaded through the first and second grasper 502 and 503 and into the cannula 501.

The tissue retractor assembly 500 of FIG. 19 is based upon a 5 mm cannula 501 commonly used in the design of laparoscopic surgical tools. The cannula 501 deploys the first and second grasper 502 and 503, respectively, by pushing them sequentially out of the distal tip of the cannula 501. The first and second grasper 502 and 503 would be pushed forward by a rod or cannula 501 sliding axially and with a force supplied by a screw or gear driven mechanism (not shown). The first and second grasper 502 and 503 may be fabricated from metal, plastic or a combination of materials that are formed in either a “C” or “U” shape which is normally closed, i.e., Raney type clips. The first and second grasper 502 and 503 would have a first and second back span 505 and 506, respectively, for guiding or attaching a length of suture 504. The first and second grasper 502 and 503 may additionally have gripping features, i.e., teeth, points, chevrons, 502a, 502b, 503a and 503b, at the open tips or on the inside surface to aid in gripping tissue. The first and second grasper 502 and 503 for the organ 507 may further be coated with a rubber, have surface features or a shape that is advantageous to grasping without damaging the organ 507. Additionally, the second grasper 503, which is to be attached to the abdominal wall 509, may have more aggressive gripping features 503a and 503b, i.e., aggressive teeth or sharp points, to attach to the abdominal wall 509. The first grasper 502, however, which is to be used to grasp the organ 507, may have atraumatic teeth at the gripping features 502a and 502b, in order to prevent damage to the organ 507.

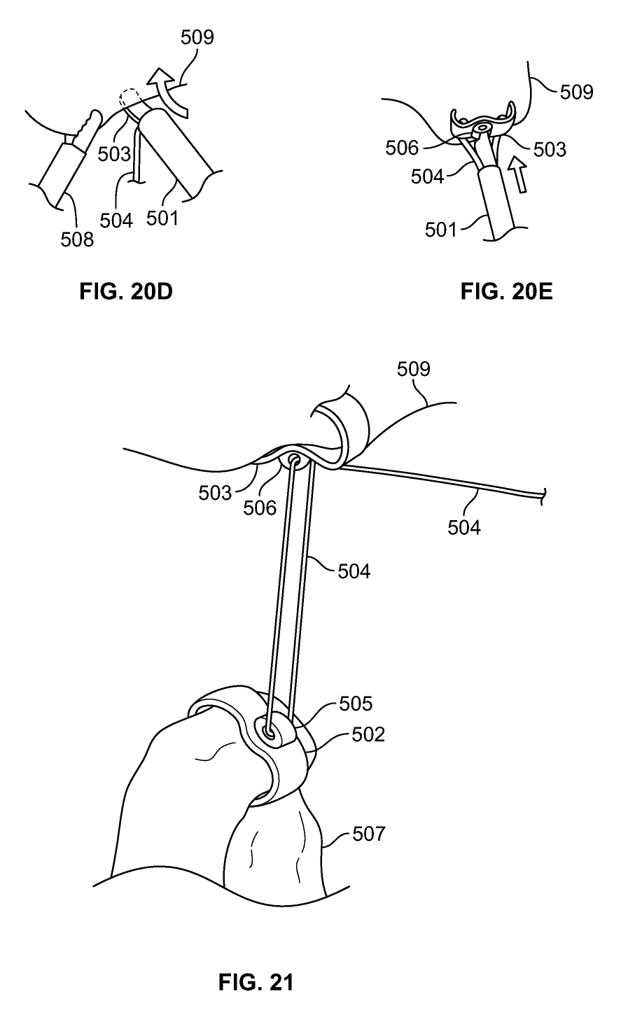

Still with reference to FIG. 19 and further with reference to FIGS. 20A-E, the tissue retractor assembly 500 is depicted at progressive steps of securing the first grasper 502 to the organ 507 and securing the second grasper 503 to the abdominal wall 509 after the tissue retractor assembly 500 has been introduced into an access port (not shown). The first and second grasper 502 and 503, as they reside in the cannula 501, would be fully opened such that they are nearly flat. The first and second grasper 502 and 503 are loaded into the cannula 501 for purposes of introduction into an access port. Once inside the port and at the organ 507 to be grasped, the first and second grasper 502 and 503 may be deployed. Generally, the clinician has a multipurpose use 5 mm grasper 508, depicted in FIG. 20A, in the surgical field during the procedure. The 5 mm grasper 508 manages the tissue of the organ 507 in question. The distal end of the cannula 501 is placed near the organ 507 and the first grasper 502 is pushed out of the cannula 501 distally by a rod or shaft which is driven by a screw or gear mechanism (not shown). The first grasper 502 will be pushed out approximately halfway to allow the clinician to position the first grasper 502 and then the first grasper 502 would be deployed. The shape of the cannula 501 and features at the tip would help to manage the dynamic nature of the first grasper 502 deployment. This design also offers the possibility of deploying multiple graspers onto the organ 507 as necessary (not shown). The cannula 501 trails a length of suture 504 that is tethered to the first grasper 502 placed on the organ 507.

Further with reference to FIGS. 20A-E, the second grasper 503 is deployed next to allow the organ 507 to be retracted. The second grasper 503 may have the same overall shape and function as the first grasper 502. The second grasper 503 may include more aggressive features on the gripping features 503a and 503b of the insufflated abdominal wall 509. The gripping features 503a and 503b may also be sharpened to the pint of forming penetrating features. The cannula 501 is advanced to the abdominal wall 509 and the general use 5 mm grasper 508 is used to manage the tissue of the abdominal wall 509. The second grasper 503 is deployed in a substantially similar method as the first grasper 502 attached to the organ 507.

With reference to FIG. 21, the exemplary first grasper 502 and second grasper 503 are depicted in operation. The cannula 501 is retracted from an access port trailing the length of suture 504 which keeps all the components tethered and allows the clinician to retract the organ 507 by increasing the tension on the length of suture 504. The length of suture 504 may be secured outside the port with a clamp or other appropriate means. At the end of the surgery, the first grasper 502 on the organ 507 may be removed with the organ 507 (in the case of a gall bladder removal). Depending on the geometry of the second grasper 503, removal of the second grasper 503 may require a specific tool which would be integrated into the cannula 501 or be a separate tool. If integrated into the cannula 501, the tool could be reintroduced to engage the second grasper 503 in order to remove it without damaging the tissue of the abdominal wall 509. Both the first and second grasper 502 and 503 may be removed through the abdominal incision created by the introduction of the access port.

Although the present disclosure has been described with reference to exemplary embodiments and implementations, it is to be understood that the present disclosure is neither limited by nor restricted to such exemplary embodiments and/or implementations. Rather, the present disclosure is susceptible to various modifications, enhancements and variations without departing from the spirit or scope of the present disclosure. Indeed, the present disclosure expressly encompasses such modifications, enhancements and variations as will be readily apparent to persons skilled in the art from the disclosure herein contained.

1. A tissue retractor assembly, comprising:
a. a cannula defining an axis and a distal end,
b. a grasper releasably engaged with respect to the distal end of the cannula, the grasper including (i) first and second legs for gripping tissue, and (ii) a locking ring for releasably locking the first and second legs relative to each other;
c. an anchor releasably engaged with respect to the distal end of the cannula, the anchor including a body and at least two sharpened legs that are adapted to move between a first position and a second anchoring position; and
d. a suture in cooperative association with the anchor and the grasper to facilitate movement of the grasper relative to the anchor.

2. The tissue retractor assembly of claim 1, wherein the at least two sharpened legs are fabricated from stainless steel.

3. The tissue retractor assembly of claim 1, wherein the at least two sharpened legs are delivered to an anatomical location in the first position and the at least two sharpened legs automatically move to the second position upon release from the distal end of the cannula.

4. The tissue retractor assembly of claim 1, wherein one or more of the at least two sharpened legs include serrations on a distal surface thereof.

5. The tissue retractor assembly of claim 1, wherein one or more of the at least two sharpened legs include rubber coating for atraumatically grasping tissue.

6. The tissue retractor assembly of claim 1, wherein the locking ring is adapted for axial translation relative to the first and second legs of the grasper.

7. The tissue retractor assembly of claim 1, wherein the anchor includes two sharpened legs that, when moved into the second position, are substantially transversely oriented relative to the axis of the cannula.

8. The tissue retractor assembly of claim 1, wherein the suture is secured relative to the grasper and passes through the anchor.

9. The tissue retractor assembly of claim 8, wherein proximal movement of the suture relative to the anchor tensions the grasper.

10. A tissue retractor assembly, comprising:
a. a cannula defining an axis and a distal end;
b. an anchor releasably engaged with respect to the distal end of the cannula, the anchor including (i) an outer tube and (ii) a central shaft that includes at least two barbs which are configured to be outwardly deployed when the central shaft is translated proximally relative to the cannula;
c. a wire form releasably engaged with respect to the distal end of the cannula, the wire form including a coiled spring that is adapted to expand radially outward;
d. a suture in cooperative association with the anchor and the wire form to facilitate movement of the wire form relative to the anchor.

11. The tissue retractor assembly of claim 10, further comprising a grasper that is configured and dimensioned to extend out of the distal end of the cannula and through the wire form.

12. The tissue retractor assembly of claim 10, wherein the wire form includes surface roughness along an interior surface thereof to enhance gripping functionality.

13. The tissue retractor assembly of claim 10, wherein the wire form includes one or more barbs along an interior surface thereof to enhance gripping functionality.

14. A tissue retractor assembly, comprising:
a. a cannula defining an axis and a distal end;
b. a grasper adapted to extend from the distal end of the cannula, the grasper including a loop of suture and a one way locking toggle;
c. an anchor releasably engaged with respect to the distal end of the cannula, the anchor including (i) a back span, (ii) one or more torsion springs, (iii) an axial connection between the back span and the one or more torsion springs, and (iv) at least two sharpened legs; and
d. a suture in cooperative association with the anchor and the grasper to facilitate movement of the grasper relative to the anchor.

15. The tissue retractor assembly of claim 14, wherein the loop of suture includes one or more cuts or barbs to increase frictional functionality of the loop of suture.

16. The tissue retractor assembly of claim 14, wherein the one way locking toggle includes a molded plastic element which allows the loop of suture to be pulled through in one direction and prevents the loop of suture from loosening thereafter.

17. The tissue retractor assembly of claim 14, wherein the anchor is defined by a substantially symmetrical structure.

18. A tissue retractor assembly, comprising:
a. a cannula defining an axis and a distal end;
b. a first grasper detachably engaged relative to the distal end of the cannula, the first grasper including a first clip configured and dimensioned to be deployed axially from a distal end of the cannula to define a first C-shaped form;
c. a second grasper detachably engaged relative to the distal end of the cannula, the second grasper including a second clip configured and dimensioned to be deployed axially from a distal end of the cannula to define a second C-shaped form; and
d. a suture in cooperative association with the first grasper and the second grasper to facilitate movement of the first grasper relative to the second grasper.

19. The tissue retractor assembly of claim 18, wherein the first clip and second clip are fabricated from a material selected from the group consisting of metal, plastic or a combination of metal and plastic.

20. The tissue retractor assembly of claim 18, wherein at least one of the first clip and the second clip include teeth at an open tip thereof to aid in gripping functionality.

21. The tissue retractor assembly of claim 18, wherein at least one of the first clip and the second clip include teeth on an inside surface thereof to aid in gripping functionality.

22. The tissue retractor assembly of claim 18, wherein at least one of the first clip and the second clip include rubber coating to aid in atraumatic grasping functionality.
